(19) United States
(12) Patent Application Publication
(10) Pub. No.: US 2023/0390489 A1
(43) Pub. Date: Dec. 7, 2023

(54) RESERVOIR ASSEMBLY AND DRUG SOLUTION INJECTION DEVICE COMPRISING SAME

(71) Applicant: EOFLOW CO., LTD., Seongnam-si Gyeonggi-do (KR)

(72) Inventors: Sangjin HAN, Gunpo-si Gyeonggi-do (KR); Hyunduk ROH, Seoul (KR); Dong Kuk OH, Seoul (KR); Kyung Seo CHO, Yongin-si Gyeonggi-do (KR)

(73) Assignee: EOFLOW CO., LTD., Seongnam-si Gyeonggi-do (KR)

(21) Appl. No.: 18/237,040

(22) Filed: Aug. 23, 2023

Related U.S. Application Data
(63) Continuation of application No. PCT/KR2022/002098, filed on Feb. 11, 2022.

(30) Foreign Application Priority Data
Feb. 25, 2021 (KR) 10-2021-0025696
Dec. 20, 2021 (KR) 10-2021-0182919

(51) Int. Cl. A61M 5/145; A61M 5/168

(52) U.S. Cl. CPC A61M 5/1456; A61M 5/1684; A61M 2205/33; A61M 2202/04

(57) ABSTRACT

Provided are a reservoir assembly and a medical liquid injection device. The reservoir assembly includes a reservoir including a storage space for medical liquid, a plunger inserted into the reservoir and moving along the reservoir, a connector connected to the plunger and including at least a portion extending to an opposite side of the storage space, a guide member into which the connector is inserted and which guides movement of the connector, and a sensor unit installed on the guide member and sensing movement of the connector.

### CROSS-REFERENCE TO RELATED APPLICATIONS

The present application is a continuation of International Application No. PCT/KR2022/002098 filed on Feb. 11, 2022, which claims the benefit of Korean Patent Applications No. 10-2021-0025696 filed on Feb. 25, 2021, and Korean Patent Applications No. 10-2021-0182919 filed on Dec. 20, 2021, in the Korean Intellectual Property Office, the entire disclosure of each of which is incorporated herein by reference.

### TECHNICAL FIELD

The present disclosure relates to a reservoir assembly and a medical liquid injection device including the same.

### BACKGROUND ART

Generally, a medical liquid injection device such as an insulin injection device is used to inject medical liquid into a body of a patient. Such a medical liquid injection device is used by medical professionals like doctors or nurses, but in most cases, by ordinary persons like patients or carers for the patients.

For diabetic patients, especially, pediatric diabetic patients, medical liquid like insulin needs to be injected into a human body at set intervals. A medical liquid injection device in the form of a patch used by being attached to a human body for a specific period has been developed, and such a medical liquid injection device may be used by being attached to the human body such as an abdomen, waist, etc., of a patient, in the form of a patch for the specific period.

To increase the effect of medical liquid injection, the medical liquid injection device needs to be controlled to precisely inject the medical liquid into the body of the patient, and it is important to precisely inject a small amount of medical liquid through a small-size medical liquid injection device.

The medical liquid injection device, when attached to the human body, needs to be comfortable to wear, convenient to use, durable, and driven with low power. In particular, the medical liquid injection device is used by being directly attached to the skin of the patient, such that it is important for a user to run the medical liquid injection device conveniently and safely.

### DISCLOSURE

### Technical Problem

The present disclosure provides a reservoir assembly that senses the amount of stored medical liquid and accurately deliver the medical liquid, and a medical liquid injection device including the reservoir assembly.

### Technical Solution

According to an aspect of the present disclosure, a reservoir assembly includes a reservoir comprising a storage space for medical liquid, a plunger inserted into the reservoir and moving along the reservoir, a connector connected to the plunger and including at least a portion extending to an opposite side of the storage space, a guide member into which the connector is inserted and which guides movement of the connector, and a sensor unit installed on the guide member and sensing movement of the connector.

### Advantageous Effects

The medical liquid injection device and the reservoir assembly according to an embodiment of the present disclosure may measure an injection amount of the medical liquid stored in the reservoir. The sensor unit may measure the amount of the medical liquid stored in the reservoir to set driving of the medical liquid injection device. The connector connected to the plunger may move together with linear movement of the plunger inside the reservoir, thus to sense the amount of the medical liquid stored in the reservoir while contacting the sensor unit or releasing the contact with the sensor unit. The connector may modify or process a shape to reinforce stiffness and flexibility, individually or simultaneously.

The medical liquid injection device and the reservoir assembly according to an embodiment of the present disclosure may accurately measure the amount of the medical liquid stored in the reservoir while reducing the total volume of the medical liquid injection device. As the connector has flexibility, the connector may move along the curved section together with movement of the plunger, thereby reducing the total volume of the medical liquid injection device.

The medical liquid injection device and the reservoir assembly according to an embodiment of the present disclosure may guide a bending direction of the connector connected to the plunger, thereby safely and accurately measuring the amount of the medical liquid. As the connector has a concave surface and a convex surface, the connector may be guided by bending in a direction. However, the scope of the present disclosure is not limited by these effects.

### DESCRIPTION OF DRAWINGS

FIG. 1 is a block diagram of a medical liquid injection system according to an embodiment of the present disclosure.

FIG. 2 is a perspective view of a medical liquid injection device according to an embodiment of the present disclosure.

FIGS. 3 to 5 show driving of a reservoir assembly according to an embodiment of the present disclosure.

FIG. 6 is an enlarged view of a region A of FIG. 4.

FIG. 7 shows a modified example of FIG. 6.

FIG. 8 shows another modified example of a reservoir assembly of FIG. 3.

FIG. 9 is a plane view of a connector of FIG. 8.

FIG. 10 shows another modified example of a reservoir assembly according to the present disclosure.

FIGS. 11 and 12 are cross-sectional views of another modified example of a reservoir assembly according to the present disclosure.

FIG. 13 shows another modified example of a reservoir assembly according to the present disclosure.

FIG. 14 is a perspective view of a driving unit and a driving module according to an embodiment of the present disclosure.

FIG. 15 shows a reservoir assembly according to another embodiment of the present disclosure.

### DETAILED DESCRIPTION

According to an aspect of the present disclosure, a reservoir assembly includes a reservoir comprising a storage space for medical liquid, a plunger inserted into the reservoir and moving along the reservoir, a connector connected to the plunger and including at least a portion extending to an opposite side of the storage space, a guide member into which the connector is inserted and which guides movement of the connector, and a sensor unit installed on the guide member and sensing movement of the connector.

The connector may be flexible.

The guide member may include a first end arranged on the reservoir to face the plunger, a second end arranged on a side surface of the reservoir, and a connection portion connecting the first end to the second end and comprising a curved section.

The sensor unit may include a plurality of contact terminals which are arranged separated from each other on the second end.

The sensor unit may include a plurality of contact terminals and generates a signal when the connector contacts the plurality of contact terminals.

At least one of the plurality of contact terminals may be arranged to rotate along linear movement of the connector.

The connector may include a concave first surface and a convex second surface arranged on an opposite side to the first surface.

The first surface may be arranged inside the curved section of the guide member, and the second surface may be arranged outside the curved section of the guide member.

According to another aspect of the present disclosure, a medical liquid injection device includes a reservoir assembly in which medical liquid is stored, a needle assembly ejecting the medical liquid, and a driving unit connected to the reservoir assembly and moving the medical liquid to the needle assembly from the reservoir in driving, in which the reservoir assembly includes a reservoir comprising a storage space for the medical liquid, a plunger inserted into the reservoir and moving along the reservoir, a connector connected to the plunger and including at least a portion extending to an opposite side of the storage space, a guide member into which the connector is inserted and which guides movement of the connector, and a sensor unit installed on the guide member and contacting the connector along movement of the connector.

### MODE FOR INVENTION

The present disclosure may have various modifications thereto and various embodiments, and thus particular embodiments will be illustrated in the drawings and described in detail in a detailed description. Effects and features of the present disclosure, and a method of achieving them will be apparent with reference to the embodiments described in detail in conjunction with the drawings. However, the present disclosure is not limited to the embodiments disclosed below, but may be implemented in various forms.

Hereinafter, embodiments will be described in detail with reference to the accompanying drawings, and in description with reference to the drawings, the same or corresponding components are given the same reference numerals, and redundant description thereto will be omitted.

In the following embodiment, singular forms include plural forms unless apparently indicated otherwise contextually.

In the following embodiments, the terms “include”, “have”, or the like, are intended to mean that there are features, or components, described herein, but do not preclude the possibility of adding one or more other features or components.

When a certain embodiment may be implemented otherwise, a particular process order may be performed differently from the order described. For example, two processes described in succession may be performed substantially simultaneously, or may be performed in an order reverse to the order described.

In the drawings, the size of components may be exaggerated or reduced for convenience of description. For example, the size and thickness of each component shown in the drawings are shown for convenience of description, and thus the present disclosure is not necessarily limited to the illustration.

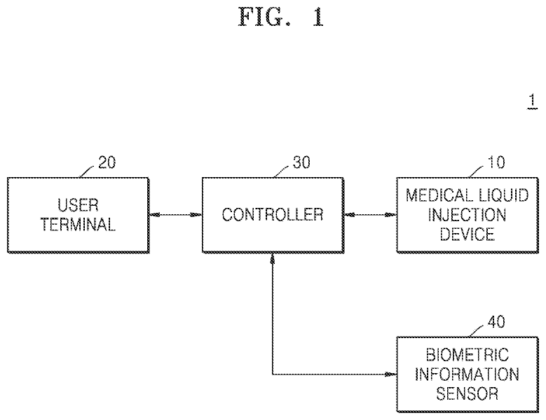

FIG. 1 is a block diagram of a medical liquid injection system 1 according to an embodiment of the present disclosure.

Referring to FIG. 1, the medical liquid injection system 1 may include a medical liquid injection device 10, a user terminal 20, a controller 30, and a biometric information sensor 40. A user may drive and control the medical liquid injection system 1 by using the user terminal 20, and periodically inject medical liquid through the medical liquid injection device 10 based on blood sugar information monitored by the biometric information sensor 40.

The medical liquid injection device 10 may perform a function to inject medical liquid to be injected to a user, e.g., insulin, glucagon, anesthetics, pain relievers, dopamine, growth hormones, smoking cessation aids, etc., based on data sensed by the biometric information sensor 40.

In addition, the medical liquid injection device 10 may transmit a device state message including remaining battery capacity information, booting success or failure, injection success or failure, etc., of the medical liquid injection device 10 to the controller 30. Messages transmitted to the controller 30 may be transmitted to the user terminal 20 through the controller 30. Alternatively, the controller 30 may transmit improved data resulting from processing the received messages to the user terminal 20.

In an embodiment, the medical liquid injection device 10 may be provided separately from the biometric information sensor 40 and may be installed separated from an object. In another embodiment, the medical liquid injection device 10 and the biometric information sensor 40 may be provided in one device.

In an embodiment, the medical liquid injection device 10 may be mounted on a user's body. In another embodiment, the medical liquid injection device 10 may be mounted on an animal to inject medical liquid.

The user terminal 20 may receive an input signal from the user to drive and control the medical liquid injection system 1. The user terminal 20 may generate a signal for driving the controller 30 to control the controller 30 to drive the medical liquid injection device 10. The user terminal 20 may also display biometric information measured from the biometric information sensor 40 and display state information of the medical liquid injection device 10.

The user terminal 20 may mean a communication terminal available in a wired/wireless communication environment. For example, the user terminal 20 may be a smartphone, a tablet personal computer (PC), a PC, a smart television (TV), a mobile phone, a personal digital assistant (PDA), a laptop, a media player, a micro server, a global positioning system (GPS) device, an electronic-book terminal, a digital broadcasting terminal, a navigation, a kiosk, an MP3 player, a digital camera, a home appliance, a camera-mounted device, and other mobile or non-mobile computing devices. The user terminal 20 may be a wearable device such as a watch, glasses, a hair band, a ring, etc., having a communication function and a data processing function. However, as described above, a terminal having installed thereon an application capable of performing Internet communication may be used without limitation.

The user terminal 20 may be one-to-one connected to the controller 30 registered in advance. The user terminal 20 may be encrypted and connected to the controller 30 to prevent the controller 30 from being driven and controlled by an external device.

In an embodiment, the user terminal 20 and the controller 30 may be separated and provided as separate devices. For example, the controller 30 may be provided to a subject having the medical liquid injection device 10 mounted thereon, and the user terminal 20 may be provided in the subject or a third party. The user terminal 20 may be driven by a carer to improve safety of the medical liquid injection system 1.

In another embodiment, the user terminal 20 and the controller 30 may be provided as one device. The controller 30 provided as one with the user terminal 20 may communicate with the medical liquid injection device 10 to control injection of the medical liquid.

The controller 30 may perform a function to transmit and receive data to and from the medical liquid injection device 10, transmit a control signal related to injection of the medical liquid such as insulin, etc., to the medical liquid injection device 10, and receive a control signal related to measurement of a biometric value such as blood sugar, etc., from the biometric information sensor 40.

The controller 30 may, for example, transmit an instruction request for measuring a user's current state to the medical liquid injection device 10 and receive measurement data from the medical liquid injection device 10 in response to the instruction request.

The biometric information sensor 40 may perform a function to measure a biometric value such as a blood sugar level, a blood pressure, a heart rate, etc., of a user according to a purpose. Data measured by the biometric information sensor 40 may be transmitted to the controller 30, and an injection period and/or an injection amount of the medical liquid may be set based on the measured data. The data measured by the biometric information sensor 40 may be transmitted to and displayed on the user terminal 20.

For example, the biometric information sensor 40 may be a sensor for measuring a blood sugar level of a subject. The biometric information sensor 40 may be a continuous glucose monitoring (CGM) sensor. The CGM sensor may be attached to the subject to continuously monitor the blood sugar level.

The user terminal 20, the controller 30, and the medical liquid injection device 10 may perform communication by using a network. For example, the network may include a local area network (LAN), a wide area network (WAN), a value added network (VAN), a mobile radio communication network, a satellite communication network, and a combination thereof, and may be a data communication network in a comprehensive sense to enable network entities to smoothly communicate with each other and include wired Internet, wireless Internet, and a mobile wireless communication network. Wireless communication may include, but not limited to, a wireless LAN (wireless fidelity, Wi-Fi), Bluetooth, Bluetooth low energy, Zigbee, Wi-Fi direct (WFD), ultra wideband (UWB), infrared Data Association (IrDA), near field communication (NFC), 5^{th}-Generation (5G), etc.

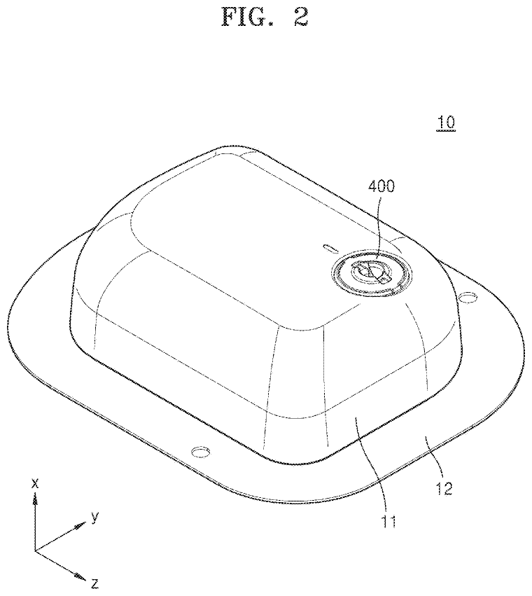

FIG. 2 is a perspective view of a medical liquid injection device according to an embodiment of the present disclosure.

Referring to FIG. 2, the medical liquid injection device 10 may be attached to the user to which the medical liquid is to be injected, and inject the medical liquid stored therein into the user in a set required amount.

The medical liquid injection device 10 may be used for various purposes according to a type of the medical liquid to be injected. For example, the medical liquid may include insulin-based medical liquid for diabetic patients, and other various types of medical liquid such as medical liquid for pancreas, medical liquid for heart, etc.

An example of the medical liquid injection device 10 may include a housing 11 covering an outer side of the medical liquid injection device 10 and an attachment portion 12 positioned adjacent to user's skin. The medical liquid injection device 10 may include a plurality of components arranged in an inner space between a reservoir 110 and the attachment portion 12. A separate bonding means may be further provided between the attachment portion 12 and the user's skin, and the medical liquid injection device 10 may be fixed to the skin by the bonding means.

The medical liquid injection device 10 may include a reservoir assembly 100, a driving unit 200, a driving module 300, a needle assembly 400, a battery, etc.

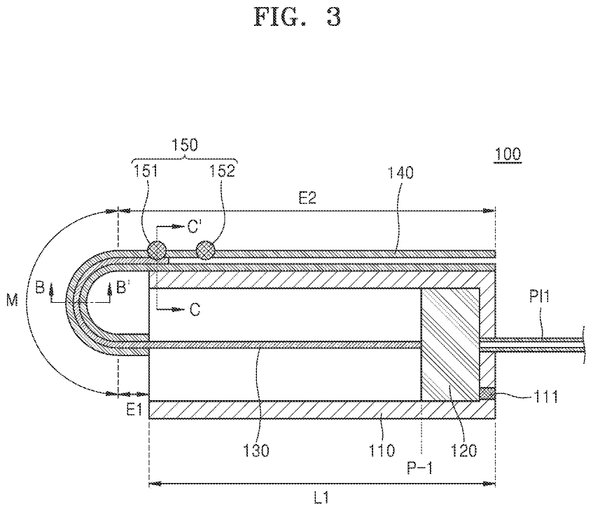

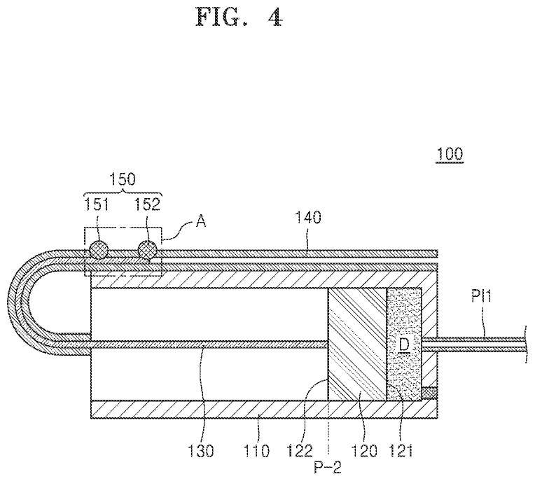

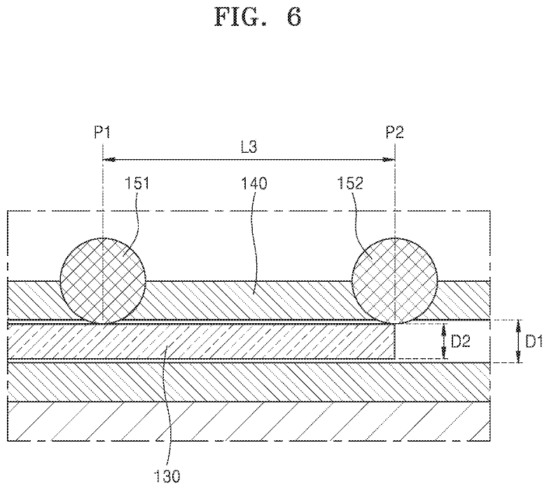

FIGS. 3 to 5 show driving of a reservoir assembly according to an embodiment of the present disclosure, and FIG. 6 is an enlarged view of a region A of FIG. 4.

Referring to FIGS. 3 to 6, the reservoir assembly 100 may include the reservoir 110, a plunger 120, a connector 130, a guide member 140, and a sensor unit 150. The reservoir assembly 100 may store medical liquid in an inner space thereof, and discharge the medical liquid in a required amount to the needle assembly 400 with a driving force generated by operations of a driving unit 200 and a driving module 300 described below.

The reservoir 110 may have an inner space in which medical liquid D is stored. A part of the inner space of the reservoir 110 may be defined as a storage space for the medical liquid D, and the storage space may be covered with the reservoir 110 and the plunger 120.

A first conduit PI1 may be connected to a side of the reservoir 110 and may extend to the driving unit 200. The medical liquid D may be discharged from the storage space to the first conduit PI1.

The reservoir 110 may have an inlet 111 through which the medical liquid D may be injected. A packing member may be arranged in the inlet 111, and an injecting needle (not shown) may be inserted into the packing member to inject the medical liquid D.

The plunger 120 may be inserted into the reservoir 110 and move in a length direction of the reservoir 110. The medical liquid D may be stored in a space covered with the plunger 120 and the reservoir 110, and the plunger 120 may move as the stored amount of the medical liquid D changes.

A side 121 of the plunger 120 may form the storage space for the medical liquid D and may contact the medical liquid D when the medical liquid D is stored in the reservoir assembly 100. Another side 122 of the plunger 120 may be connected to the connector 130, such that the connector 130 may move together with movement of the plunger 120.

The plunger 120 may closely contact an inner surface of the reservoir 110 to prevent the stored medical liquid D from leaking. For example, the plunger 120 may be manufactured with a material having elasticity such as rubber, silicon, etc., to closely contact the inner surface of the reservoir 110.

The connector 130 may be connected to the plunger 120 to move together with movement of the plunger 120. At least a part of the connector 130 may extend to the opposite side of the storage space of the reservoir 110.

The connector 130 may not be limited to a specific shape and may have various shapes. For example, the connector 130 may be configured variously such as in a thin band form, a wire form, etc. However, for convenience of a description, the following description will be made based on an example where the connector 130 has a thin band form.

The connector 130 may modify or process a shape to reinforce stiffness and flexibility, individually or simultaneously.

At least a part of the connector 130 may include a conductive material. In particular, the connector 130 may include a conductive material on an outer region thereof. As the connector 130 includes a conductive material, the connector 130 may be electrically connected to the sensor unit 150.

The connector 130 may be formed flexibly. The connector 130 may have predetermined flexibility so as to be bent by an external force. As the connector 130 has flexibility, the connector 130 may move along the guide member 140 having a curved section.

In an embodiment, the connector 130 may be formed of a metal material. The connector 130 may have a form of a metal strip and have predetermined stiffness and flexibility. As the connector 130 has conductivity, the connector 130 may electrically connect a first contact end 151 to a second contact end 152 by contacting the first contact end 151 and the second contact end 152.

The connector 130 may be formed to have a length greater than or equal to a first length L1 in an axial direction of the reservoir 110. Referring to FIG. 3, at an initial position before injection of the medical liquid D, the connector 130 may be in a state of being inserted into the guide member 140. In particular, as an end of the connector 130 is inserted into the curved section of the guide member 140, a length of the connector 130 may be greater than or equal to the first length L1 in the axial direction of the reservoir 110.

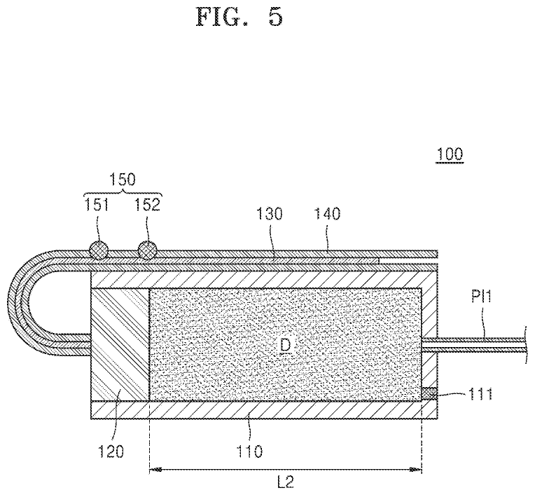

More specifically, the length of the connector 130 may be greater than the first length L1 of the medical liquid D in the axial direction of the reservoir 110 when the medical liquid D is fully stored in the reservoir 110. Referring to FIG. 5, when the medical liquid D is fully stored in the reservoir 110, a second length L2 may be formed in the axial direction and may correspond to a range where the plunger 120 may move. The plunger 120 may move in the axial direction of the reservoir 110 by a length corresponding to the second length L2, and the length of the connector 130 may be greater than the second length L2 as a part of the connector 130 is inserted into the guide member 140 arranged outside the reservoir 110 at the initial position.

As the length of the connector 130 is greater than or equal to the first length L1 or is greater than the second length L2, the connector 130 may stably maintain the initial position. As the end of the connector 130 keeps a state of being inserted into the guide member 140, the connector 130 may stably move along the guide member 140.

The connector 130 may be inserted into the guide member 140 which may guide movement of the connector 130. The guide member 140 may guide movement of the connector 130 along a set path by movement of the plunger 120.

The guide member 140 may be connected to the reservoir 110. The guide member 140 may be arranged on an outer side of the reservoir 110 and at least a part thereof may extend along the reservoir 110. The guide member 140 may have the curved section. Through the curved section of the guide member 140, the connector 130 may move along a curved path.

In an embodiment, the guide member 140 may include a first end E1, a second end E2, and a connection portion M. The first end E1 may be arranged in the reservoir 110 to face the plunger 120. The second end E2 may be arranged on a side surface of the reservoir 110. The connection portion M may connect the first end E1 to the second end E2.

The first end E1 and the second end E2 may correspond to linear sections, and the connection portion M may correspond to a curved section. A length of the first end E1 may be less than that of the second end E2, and the first end E1 and the second end E2 may be arranged in parallel to each other.

The first end E1 may guide the connector 130 coming from the reservoir 110 to be stably inserted into the guide member 140. As the first end E1 has the linear section, the first end E1 may buffer a sudden change in direction while guiding the connector 130. That is, as the connector 130 passes through the linear section before entering the curved section, a direction of the connector 130 coming from the reservoir 110 is not immediately changed. Once the connector 130 is inserted into the guide member 140, the connector 130 enters the curved section after passing through at least a partial linear section, such that the guide member 140 may stably guide movement of the connector 130.

The second end E2 may extend in the length direction of the reservoir 110. As the second end E2 extends along a sidewall of the reservoir 110, a volume of the reservoir assembly 100 may be minimized. Moreover, the sensor unit 150 arranged at the second end E2 may supported integrally with the reservoir 110 and the guide member 140, thus being stably fixed.

The connection portion M may form the curved section to change the movement direction of the connector 130. In particular, the connection portion M may connect the first end E1 and the second end E2 that are parallel to each other, thus changing the movement direction of the connector 130.

The guide member 140 may have a gap D1 greater than a thickness D2 of the connector 130. As the thickness D2 of the connector 130 is greater than the gap D1 of the guide member 140, the connector 130 may be easily inserted into the guide member 140.

The sensor unit 150 may measure driving of the medical liquid injection device 10. The sensor unit 150 may measure the amount of medical liquid stored in the reservoir 110, or measure driving of the driving module 300, driving of the driving unit 200, a movement distance of the plunger 120, etc.

The sensor unit 150 may sense movement of the connector 130. The sensor unit 150 may be installed to contact the connector 130. The sensor unit 150 may measure movement of the connector 130 to calculate the amount of the medical liquid D stored in the reservoir 110.

The sensor unit 150 may be formed with various sensors that sense a position of a component. For example, the sensor unit 150 may have various forms such as an optical sensor, a contact sensor, etc. However, for convenience of a description, the description will be made mainly of an example where the sensor unit 150 is a contact sensor that measures a position through contact by the connector 130.

The sensor unit 150 may include a plurality of contact terminals. When the connector 130 contacts the plurality of contact terminals, the connector 130 may generate a signal. The contact terminal may measure electrical contact to measure each event or data. Each contact terminal may be connected to a control module that is a circuit board.

In an embodiment, the sensor unit 150 may include a first contact terminal 151 and a second contact terminal 152. The first contact terminal 151 and the second contact terminal 152 may be arranged separated from each other, and the connector 130 may contact the first contact terminal 151 and/or the second contact terminal 152 by linearly moving.

The connector 130 may contact the first contact terminal 151 at a first position P1 and contact the second contact terminal 152 at a second position P2.

The plurality of contact terminals may be arranged on the guide member 140. The plurality of contact terminals may be arranged at the second end E2 of the guide member 140. As the second end E2 is connected to the reservoir 110, the plurality of contact terminals may be formed integrally with the reservoir 110 and the guide member 140, thus providing structural stability.

As the plurality of contact terminals are arranged at the second end E2, they may stably contact the connector 130. The first end E1 may have a short section length, thus failing to sufficiently securing a space where the plurality of contact terminals are to be arranged, and the connection portion M may have the curved section, making it difficult to stably maintain contact. The second end E2 may secure a space where the plurality of contact terminals are to be arranged.

The plurality of contact terminals may be arranged separated from each other. For example, the first contact terminal 151 and the second contact terminal 152 may be separated from each other by a preset distance L3. The separation distance L3 between the first contact terminal 151 and the second contact terminal 152 may be set as a criterion for measuring a change of the medical liquid D stored in the reservoir 110.

The first contact terminal 151 may be arranged at the second end E2 and in adjacent to the connection portion M. The first contact terminal 151 may be arranged in adjacent to the curved section to easily contact the connector 130. The connector 130 may have flexibility and stiffness, such that the connector 130 passing through the curved section may be spread outwardly. As the first contact terminal 151 is arranged at the second end E2 adjacent to the connection portion M, the connector 130 passing through the connection portion M may easily contact the first contact terminal 151 and continuously maintain the contact with the first contact terminal 151.

In an embodiment, the plurality of contact terminals may be arranged to be exposed from the guide member 140. The plurality of contact terminals may be exposed from an inner surface of the guide member 140 to contact the connector 130. The first contact terminal 151 and the second contact terminal 152 may be exposed from the inner surface of the guide member 140 to contact a surface of the connector 130.

In an embodiment, the plurality of contact terminals may be arranged to protrude from a surface of the guide member 140. The plurality of contact terminals may protrude from the inner surface of the guide member 140 to contact the connector 130. The first contact terminal 151 and the second contact terminal 152 may be exposed from the inner surface of the guide member 140 to contact a surface of the connector 130.

<Process of Medical Liquid being Injected into Reservoir>

In a sequential order of FIGS. 3 to 5, the medical liquid D may be stored in the reservoir 110. In a process of the medical liquid D being injected into the reservoir 110, the connector 130 may first contact the first contact terminal 151 at a first position P1 (the plunger 120 at a position P-1), and then the connector 130 may contact the second contact terminal 152 at a second position P2 (the plunger 120 at a position P-2).

In an embodiment, the connector 130 may electrically connect the first contact terminal 151 to the second contact terminal 152. When the first contact terminal 151 and the second contact terminal 152 are electrically connected to each other through the connector 130, then the controller may recognize a particular event of the reservoir assembly 100.

For example, when the connector 130 contacts the first contact terminal 151 and the second contact terminal 152, the sensor unit 150 may sense that medical liquid stored in the reservoir 110 is stored in a first reference amount (e.g., 10%, 20%, 30%, etc.).

When the controller recognizes that the medical liquid D is stored in the reservoir 110 in the set first reference amount, then the controller may awake the medical liquid injection device 10 in a first mode. That is, the controller may identify that a certain amount of medical liquid is stored in the reservoir 110, and start certain driving to pre-heat the medical liquid injection device 10.

In another embodiment, the connector 130 may contact at least one of contact terminals of the sensor unit 150 to generate an electrical signal. The controller may recognize a first event upon contacting the first contact terminal 151, and recognize a second event upon contacting the second contact terminal 152.

For example, the connector 130 may awake the medical liquid injection device 10 by contacting the first contact terminal 151 and sense the amount of medical liquid stored in the medical liquid injection device 10 by contacting the second contact terminal 152.

For example, the connector 130 may primarily sense the amount of medical liquid stored in the reservoir 110 while awaking the medical liquid injection device 10 by contacting the first contact terminal 151, and secondarily sense the amount of medical liquid stored in the medical liquid injection device 10 by contacting the second contact terminal 152.

Thereafter, the controller may inject the medical liquid into a patient in a second mode.

<Process of Medical Liquid being Discharged from Reservoir to Needle>

In a reverse order of FIGS. 3 to 5, the medical liquid D may be discharged from the reservoir 110 to a needle assembly. In a process of the medical liquid D being discharged to a needle N, the connector 130 may first contact the second contact terminal 152 at the second position P2 (the plunger 120 at the position P-2), and then contact the first contact terminal 151 at the first position P1 (the plunger 120 at the position P-1).

In an embodiment, the controller may activate a third mode when the sensor unit 150 is electrically released. When the connector 130 maintains contact between the first contact terminal 151 and the second contact terminal 152 and then releases the contact of the second contact terminal 152, electrical connection between the first contact terminal 151 and the second contact terminal 152 may be released. When the electrical connection between the first contact terminal 151 and the second contact terminal 152 is released, then the controller may recognize a particular event of the reservoir assembly 100.

In the third mode, the controller may transmit an alarm signal indicating that the amount of medical liquid stored corresponds to a second reference amount, to the user through the user terminal 20, the controller 30, and/or an alarm unit (not shown).

The second reference amount may be defined as the amount of medical liquid set by the controller at the time of driving the third mode. The controller may transmit, to the user, information indicating that the amount of medical liquid remaining in the reservoir 110 is the preset second reference amount, thereby allowing the user to prepare for replacing the medical liquid injection device 10.

In an embodiment, the first reference amount may be set as the amount of medical liquid stored that is equal to the second reference amount. That is, the second reference amount may be set equal to the amount of the medical liquid D actually stored in the reservoir 110. When the connector 130 contacts the second contact terminal 152 or releases the contact with the second contact terminal 152 by forward or backward movement of the plunger 120, a position of the plunger 120 in the reservoir 110 is the same, such that the first reference amount and the second reference amount may be set equal to each other.

In another embodiment, the first reference amount may be set as the amount of medical liquid stored that is greater than the second reference amount. The first reference amount may be a reference value set for driving of the first mode, and may be set substantially equal to the amount of medical liquid stored in the reservoir 110. However, the second reference amount may be the amount of medical liquid recognized by the controller at the start of the third mode, and may be set less than the amount of medical liquid actually remaining in the reservoir 110 to have a margin.

As the second reference amount is set less than the amount of medical liquid actually stored in the reservoir 110, the reservoir 110 may have a margin corresponding to a difference between the actual residual amount of the medical liquid and the second reference amount. While the medical liquid injection device 10 notifies that there is no remaining medical liquid, the medical liquid remaining in the reservoir 110 may be further used, thereby eliminating sudden disconnection of the medical liquid or an accident and thus improving the safety of the medical liquid injection device 10.

As the residual amount of the medical liquid is important in the third mode, the controller may precisely calculate the injection amount of the medical liquid and the residual amount of the medical liquid remaining in the reservoir 110, in the third mode. In the third mode, the controller may accurately measure a rotation angle of the driving unit 200 and a movement distance of the plunger 120, based on data obtained from an encoder, etc., thereby precisely calculating the ejection amount of the medical liquid and the residual amount of the medical liquid remaining in the reservoir 110. The residual amount of the medical liquid, accurately calculated in the third mode, may be transmitted to the user in real time, allowing the user to recognize any possible danger.

In an embodiment, the medical liquid injection device 10 may accurately count the amount of the medical liquid remaining in the reservoir 110, in the third mode. In the second mode, the amount of the medical liquid stored in the reservoir 110 falls out of a preset range (i.e., the second reference amount) in the second mode, such that the amount of the medical liquid remaining in the reservoir 110 is not precisely counted, but in the third mode, the amount of the medical liquid stored in the reservoir 110 may be counted as a required amount. The amount of the medical liquid stored in the medical liquid injection device 10 may be precisely counted when the stored amount is at a level requiring notification, thereby reducing a control load of the medical liquid injection device 10.

In another embodiment, the connector 130 may release the contact with at least one of the contact terminals of the sensor unit 150 to recognize a different event. The controller may recognize a third event when the connector 130 releases the contact with the second contact terminal 152, and the controller may recognize a fourth event when the connector 130 releases the contact with the first contact terminal 151.

For example, the controller may transmit an alarm signal to the user when the connector 130 releases the contact with the second contact terminal 152, and the controller may forcedly terminate the medical liquid injection device 10, continuously generate an alarm signal to the user terminal 20, or reduce the amount of the medical liquid to be injected to the user, or lengthen an injection period when the connector 130 releases the contact with the first contact terminal 151.

The medical liquid injection device 10 and the reservoir assembly 100 according to an embodiment of the present disclosure may measure the injection amount of the medical liquid stored in the reservoir 110. The sensor unit 150 may measure the amount of the medical liquid stored in the reservoir 110, thereby setting driving of the medical liquid injection device 10. When the plunger 120 linearly moves inside the reservoir 110, the connector 130 connected to the plunger 120 also moves together to sense the amount of the medical liquid stored in the reservoir 110 while contacting or releasing the contact with the sensor unit 150.

The medical liquid injection device 10 and the reservoir assembly 100 according to an embodiment of the present disclosure may accurately measure the amount of the medical liquid stored in the reservoir 110 while reducing the total volume of. As the connector 130 has flexibility, the connector 130 may move along the curved section together with movement of the plunger 120, thereby reducing the total volume.

The medical liquid injection device 10 and the reservoir assembly 100 according to an embodiment of the present disclosure may be pre-heated to improve driving efficiency when the medical liquid is filled to some extent in the reservoir 110. When the sensor unit 150 senses that the amount of the medical liquid to be injected to the reservoir 110 is greater than or equal to or exceeds the first reference amount, the medical liquid injection device 10 may prepare for driving of some parts in the first mode and inject the medical liquid immediately upon being attached onto the user.

When the medical liquid stored in the reservoir 110 is below a predetermined range, the medical liquid injection device 10 and the reservoir assembly 100 according to an embodiment of the present disclosure may sense so and indicate so to the user. When the sensor unit 150 senses that the amount of the medical liquid stored in the reservoir 110 is reduced less than or equal to or less than the second reference amount, the medical liquid injection device 10 may precisely count the amount of the medial liquid remaining in the reservoir 110 and transmit information about the same to the user.

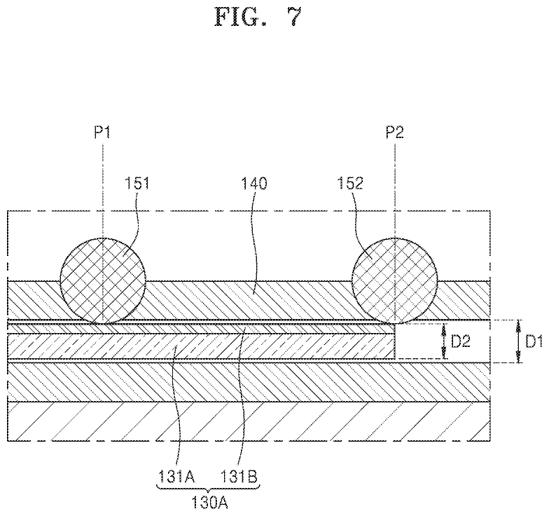

FIG. 7 shows a modified example of FIG. 6.

Referring to FIG. 7, a connector 130A may include a base portion 131A and a conductive layer 132A. The conductive layer 132A may cover a surface of the base portion 131A. The conductive layer 132A may be formed of a material having electric conductivity.

For example, the connector 130A may form the conductive layer 132A by coating a conductive material on the base portion 131A that is a flexible printed circuit board (FPCB). The conductive layer 132A may contact the first contact terminal 151 and the second contact terminal 152. Thus, when the connector 130A moves, the conductive layer 132A may contact the sensor unit 150 to measure the stored amount of the medical liquid.

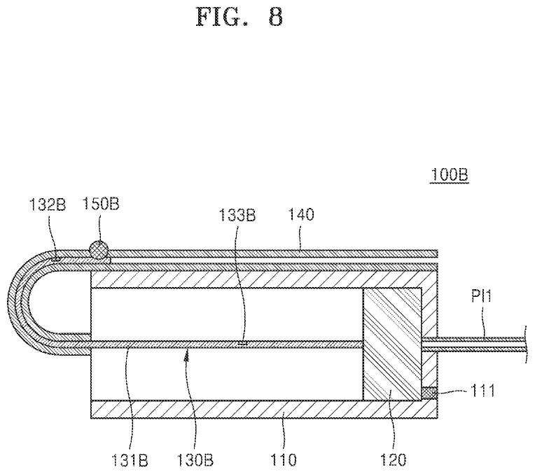

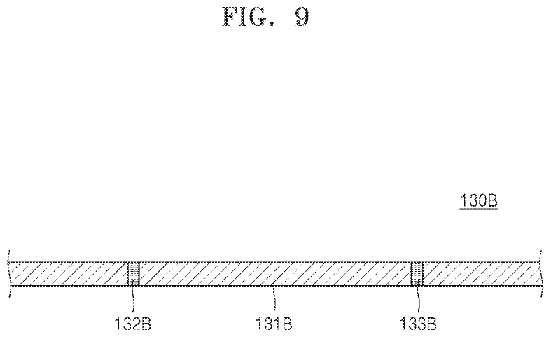

FIG. 8 is a view showing another modified example of the reservoir assembly of FIG. 3, and FIG. 9 is a plane view showing the connector of FIG. 8.

Referring to FIGS. 8 and 9, a reservoir assembly 100B may include the reservoir 110, the plunger 120, a connector 130B, the guide member 140, and a sensor 150B.

The connector 130B may include a base portion 131B and a plurality of conductive portions. For example, the plurality of conductive portions may be arranged separated from each other on the base portion 131B.

The sensor 150B may be mounted on the guide member 140. The sensor 150B may contact the conductive portion to measure the movement distance of the plunger 120 and calculate the amount of the medical liquid stored in the reservoir 110.

In the reservoir assembly 100B, one sensor 150B may be installed on the guide member 140. When the connector 130B moves, each conductive portion may contact the sensor 150B. Each conductive portion may indicate different information, such that the sensor 150B may measure the movement distance of the plunger 120 and calculate the amount of the medical liquid stored in the reservoir 110 in contact with each conductive portion.

For example, when the sensor 150B contacts a first conductive portion 132B, the controller may recognize that medical liquid of an amount A is stored in the reservoir 110. When the sensor 150B contacts a second conductive portion 133B, the controller may recognize that medical liquid of an amount B is stored in the reservoir 110.

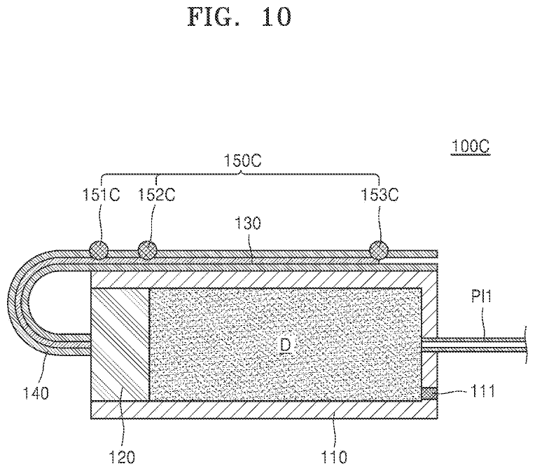

FIG. 10 shows another modified example of a reservoir assembly according to the present disclosure.

Referring to FIG. 10, a reservoir assembly 100C may include the reservoir 110, the plunger 120, the connector 130, the guide member 140, and a sensor unit 150C.

The sensor unit 150C may include a plurality of contact terminals. The sensor unit 150C may include a first contact terminal 151C, a second contact terminal 152C, and a third contact terminal 153C.

The first contact terminal 151C and the second contact terminal 152C may be substantially the same as the first contact terminal 151 and the second contact terminal 152 according to the above-described embodiment.

The third contact terminal 153C may be arranged separated from the second contact terminal 152C and measure storage of the medical liquid D.

In an embodiment, when the medical liquid D is fully stored in the reservoir 110, the third contact terminal 153C may contact the connector 130. When the connector 130 contacts the third contact terminal 153C, it may be recognized that the medical liquid D stored in the reservoir 110 corresponds to a maximum limit.

When the medical liquid D is injected through the inlet 111, the connector 130 may contact each contact terminal in an order of the first contact terminal 151C, the second contact terminal 152C, and then the third contact terminal 153C. When the third contact terminal 153C contacts the connector 130, it may be recognized that the medical liquid D is fully stored in the reservoir 110 and such state information may be transmitted to the controller. Thus, the user may stop injecting the medical liquid D and attach the medical liquid injection device 10 to the patient.

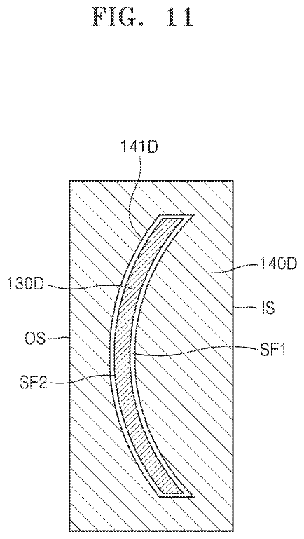

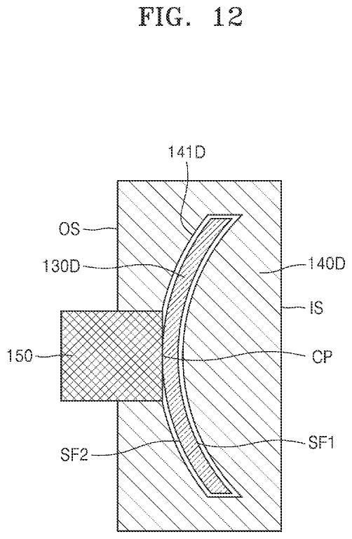

FIGS. 11 and 12 are cross-sectional views of another modified example of a reservoir assembly according to the present disclosure. FIG. 11 shows a cross-section corresponding to a region cut along B-B′ of FIG. 3, and FIG. 12 shows a cross-section corresponding to a region cut along C-C′ of FIG. 3.

Referring to FIGS. 11 and 12, the reservoir assembly may include a connector 130D having a curvature. As the flexible connector 130D of the reservoir assembly has a curvature, the connector 130D may be easily and simply bent in the curved section.

The connector 130D may have a curvature in at least some region. The connector 130D may have a concave portion and a convex portion on a cross-section thereof.

The connector 130D may have a first surface SF1 and a second surface SF2, and the second surface SF2 may be arranged on an opposite side of the first surface SF1. The first surface SF1 may have a concave shape, and the second surface SF2 may have a convex shape.

At least a part of the connector 130D may be inserted into a guide member 140D that may guide movement and bending of the connector 130D. The guide member 140D may guide bending of the connector 130D in the curved section, and guide linear movement of the connector 130D in the linear section.

The guide member 140D may include a guide path 141D that guides movement of the connector 130D. The guide member 140D may include an inner surface that is convex to correspond to the first surface SF1 of the connector 130D and an inner surface that is concave to correspond to the second surface SF2. While an embodiment is shown in the drawings where the guide path 141D has a hole shape arranged inside the guide member 140D, the guide path 141D may be set to have various shapes that guide movement of the connector 130D like a groove shape, etc., without being limited thereto.

Referring to FIGS. 3 and 11, the connector 130D may be such that the first surface SF1 is arranged on an inner side IS of the curved section of the guide member 140D and the second surface SF2 is arranged on an outer side OS of the curved section of the guide member 140D. With such arrangement, the connector 130D may be easily bent along the curved section.

When the concave first surface SF1 is arranged on the inner side IS of the curved section and the convex second surface SF2 is arranged on the outer side OS of the curved section, the connector 130D may be easily bent toward the inner side IS from the outer side OS, but bending thereof from the inner side IS to the outer side OS may be suppressed and bending thereof in a height direction of the connector 130D may be suppressed.

Referring to FIG. 12, the convexly protruding second surface SF2 of the connector 130D may contact the sensor unit 150. Preferably, the connector 130D may contact the sensor unit 150 in a central region CP thereof to sense movement of the connector 130D.

The second surface SF2 of the connector 130D protrudes outwardly, thus accurately contacting the sensor unit 150. When a surface of the connector 130D is flat, poor contact with the sensor unit may occur, but a convex part of the second surface SF2 may easily contact the sensor unit 150, thereby minimizing occurrence of the poor contact.

In another modified example, a conductive layer or a conductive portion of the above-described embodiment may be arranged on the second surface SF2 of the connector 130D, and movement of the connector 130D may be accurately sensed through contact with the conductive layer or the conductive portion.

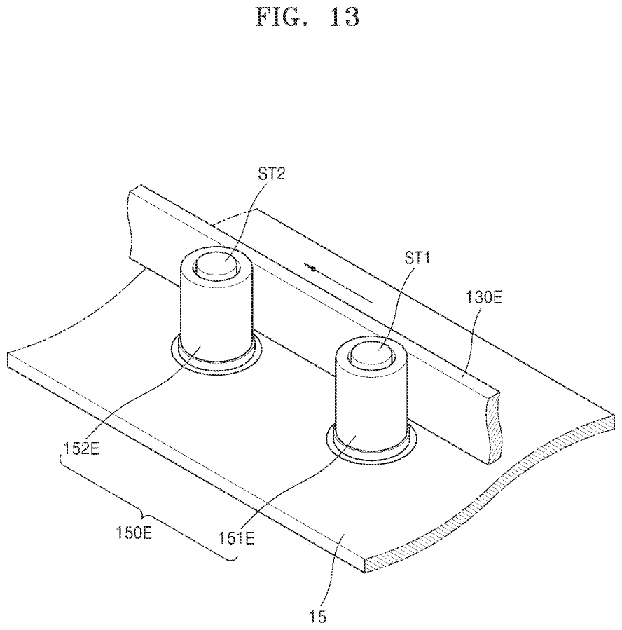

FIG. 13 shows another modified example of a reservoir assembly according to the present disclosure.

Referring to FIG. 13, a connector 130E may contact a rotatable sensor unit 150E. An outer portion of the sensor unit 150E may rotate together with linear movement of the connector 130E, such that the connector 130E may easily move.

On a support plate 15, the sensor unit 150E including a first contact terminal 151E and a second contact terminal 152E may be mounted. The support plate 15 may include a first shaft ST1 and a second shaft ST2 that protrude upwardly. The first contact terminal 151E may be rotatably mounted on the first shaft ST1, and the second contact terminal 152E may be rotatably mounted on the second shaft ST2.

When the medical liquid stored in the reservoir changes, the connector 130E may move along the guide member 140. By sensing contact between the sensor unit 150E and the connector 130E, the stored amount and the ejection amount of the medical liquid may be calculated.

The sensor unit 150E may be such that the first contact terminal 151E is rotatably mounted on the first shaft ST1 and the second contact terminal 152E is rotatably mounted on the second shaft ST2, such that the first contact terminal 151E and the second contact terminal 152E that contact the connector 130E may rotate. The sensor unit 150E may rotate while maintaining contact with the connector 130E, thereby avoiding resisting linear movement of the connector 130E. As the reservoir assembly 100 minimizes frictional resistance between the connector 130E and the sensor unit 150E, the amount of medical liquid may be accurately measured.

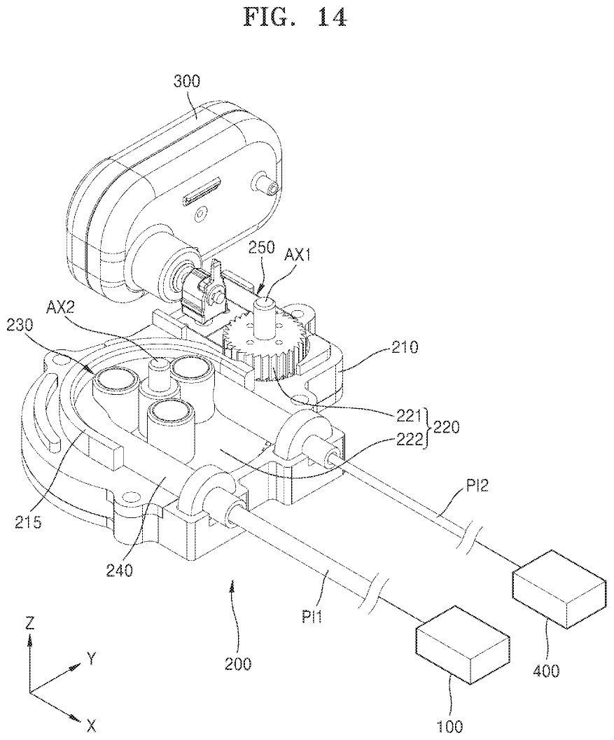

FIG. 14 is a perspective view of a driving unit and a driving module according to an embodiment of the present disclosure.

Referring to FIG. 14, the driving unit 200 may be connected to the driving module 300 and may be driven by a driving force generated in the driving module 300.

The driving unit 200 may include a base 210, a rotation unit 220, a force application unit 230, a tube 240, and a driving piece 250, and the driving module 300 may be connected to the driving piece 250.

The base 210 may support the driving unit 200 and form an exterior. At least one of the rotation unit 220, the force application unit 230, the tube 240, and the driving piece 250 may be installed and supported on the base 210.

The base 210 may include a guide portion 215. The guide portion 215 may extend along a circumferential direction of a second rotating member 222, and support the tube 240. A part of the guide portion 215 may protrude from a surface of the base 210 and extend along the curved section of the tube 240. Another part of the guide portion 215 may extend in the linear section of the tube 240.

The guide portion 215 may support force applied from the force application unit 230 to guide movement of the medical liquid. When the force application unit 230 applies force to the tube 240, the guide portion 215 may support the tube 240 on the opposite side to the force application unit 230. When the force application unit 230 applies force to the tube 240 in the curved section, the tube 240 may be compressed such that an internal cross-sectional area where the medical liquid flows is zero at a force application point where the tube 240 and a force application portion contact each other. At this time, when the force application unit 230 rotates, the medical liquid of the tube 240 moves together.

In an embodiment, the guide portion 215 may be arranged outside the curved section, and a region where the force application unit 230 applies force to the tube 240 may be arranged inside the curved section.

While an embodiment is shown in the drawings where the guide portion 215 is arranged outside the tube 240 and the force application unit 230 is arranged inside the tube 240, the present disclosure is not limited thereto, such that the guide portion may be arranged inside the tube and the force application unit may be arranged outside the tube.

The rotation unit 220 may be mounted on a side of the base 210 and rotate by receiving a driving force from the driving module 300. The rotation unit 220 may contact an end of the driving piece 250 and rotate in a direction along with linear reciprocating movement of the driving piece 250. The rotation unit 220 may be defined as a component with at least some configuration thereof rotating by receiving the driving force from the driving module 300 to rotate the force application unit 230.

In an embodiment, a plurality of members may be operatively connected in the rotation unit 220. The rotation unit 220 may include a first rotating member 221 and a second rotating member 222.

The first rotating member 221 may contact the end of the driving piece 250 to rotate along with linear reciprocating movement of the driving piece 250. The second rotating member 222 may be connected to the first rotating member 221 to rotate along with rotation of the first rotating member 221. The second rotating member 222 may have a plate shape and rotate with respect to a second axis AX2.

The ejection amount of the medical liquid may be set according to the rotation angle of the second rotating member 222. That is, the second rotating member 222 may rotate such that the medical liquid inside the tube 240 may be ejected in a required amount by the rotation angle of the force application unit 230. The rotation angle and the rotation speed of the second rotating member 222 may be set according to the number of times of movement of a driving shaft 310 and the number of tooth of the rotation unit 220.

The force application unit 230 may be mounted on the rotation unit 220 and may rotate together with the rotation unit 220. The force application unit 230 may apply force to the tube 240 while rotating with respect to the second axis AX2. The force application unit 230 may apply force to the tube 240 to compress the tube 240 at a contact point when contacting the curved section of the tube 240.

The force application unit 230 may include a plurality of rollers. When the driving unit 200 is driven, at least one force application portion may be arranged in the curved section. More preferably, when the force application unit 230 rotates, at least two force application portions may form a force application point in the curved section.

As described above, at a point where the force application portion of the force application unit 230 contacts the tube 240, the tube 240 may be compressed by the force application portion such that the internal cross-sectional area of the tube 240 may be zero. As at least two force application points are formed in the curved section, a required amount of medical liquid may be discharged according to the rotation angle of the second rotating member 222.

The tube 240 may be arranged in adjacent to the rotation unit 220, and at least a portion thereof may have a curved section extending in a circumferential direction. The tube 240 may be formed of a flexible material and thus may be compressed by the force application portion of the force application unit 230.

The tube 240 may be installed between the reservoir assembly 100 and the needle assembly 400 to pass through the rotation unit 220. A part of the tube 240 may extend in the circumferential direction of the second rotating member 222.

An end of the tube 240 may be connected to the first conduit PI1 through which the medical liquid of the reservoir 110 may move. The other end of the tube 240 may be connected to the second conduit PI2 for ejection to a needle of the needle assembly 400.

The driving piece 250 may be arranged between the driving module 300 and the rotation unit 220 to transmit the driving force generated in the driving module 300 to the rotation unit 220. The driving piece 250 may be connected to the driving shaft 310 to linearly reciprocate along with movement of the driving shaft 310.

The driving module 300 may use any type of device having a medical liquid suction force and a medical liquid ejection force by electricity.

For example, the driving module 300 may use any type of pump such as a mechanical displacement micropump, an electromagnetic motion micropump, etc. The mechanical displacement micropump may be a pump using motion of a solid or fluid such as a gear or diagram to cause a pressure difference for inducing flow of the fluid, and may include a diaphragm displacement pump, a fluid displacement pump, a rotary pump, etc. The electromagnetic motion micropump may be a pump using energy in an electric or magnetic form directly for movement of the fluid, and may include an electro hydrodynamic pump (EHD), an electro osmotic pump, a magneto hydrodynamic pump, an electro wetting pump, etc.

In another embodiment, the driving module may include a shape memory alloy (SMA). The driving module may be formed of a known shape-memory material without being limited to a particular material. For example, the driving module may be formed of an alloy of nickel and titanium.

The driving module may include a wire made of an SMA. An electrical signal may be alternately applied to the wire, such that the wire may linearly reciprocate in one direction. Depending on a direction of current applied to the wire, the wire may shrink or expand and the driving piece 250 connected to the wire may linearly reciprocate.

The medical liquid injection device 10 may discharge the medical liquid D to the needle assembly 400 from the reservoir 110 by driving of the driving unit 200. When the driving force generated in the driving module 300 is transmitted to the driving unit 200, the rotation unit 220 of the driving unit 200 may rotate and the force application unit 230 may apply force to the tube 240, thus moving the medical liquid D.

That is, the reservoir assembly 100 may not include an additional component for moving the plunger 120. By rotation of the force application unit 230, the medical liquid D may be discharged to the needle from the reservoir 110. Thus, a complex mechanism for driving the plunger 120 is not required, enabling the compact configuration of the reservoir assembly 100. Moreover, as the compact reservoir assembly 100 is provided, the total size of the medical liquid injection device 10 may be reduced.

As a specific pressure is applied to an outer side of the reservoir assembly 100, the reservoir assembly 100 may eject a required amount of medical liquid. In the reservoir assembly 100, the side 121 of the plunger 120 may contact the medical liquid and the other side 122 may communicate with outside such that an external atmospheric pressure may press the other side 122. Referring to FIG. 4, a part connected to the connector 130 in an inner space of the reservoir 110 may be connected to the outside of the reservoir 110. Thus, the part connected to the connector 130 may maintain the external atmospheric pressure that is constant at all times.

As a side of the plunger 120 is connected to the outside, the reservoir assembly 100 may maintain a constant external pressure at all times. Thus, when a constant driving force is transmitted from the driving unit 200, a required amount of the medical liquid D may be ejected to the needle.

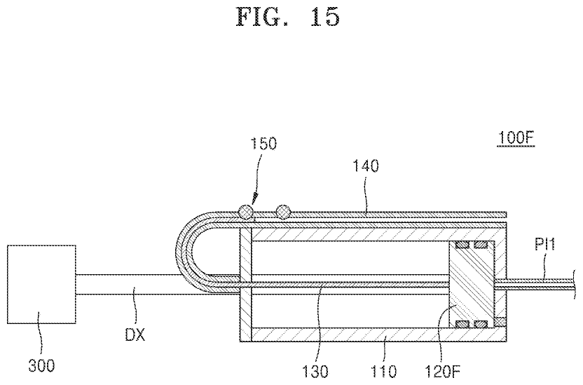

FIG. 15 shows a reservoir assembly according to another embodiment of the present disclosure.

Referring to FIG. 15, a reservoir assembly 100F may be similar to the reservoir assembly 100 according to the above-described embodiment. However, there is a difference in a driving mechanism for driving a plunger 120F, which will be mainly described.

The plunger 120F may be connected to a driving shaft DX which may receive a driving force generated in the driving module 300. When the driving force is transmitted to the driving shaft DX, the plunger 120F may move forward or backward along the reservoir 110.

The connector 130 may move along the guide member 140 together with forward or backward movement of the plunger 120F, and the sensor unit 150 may measure the amount of medical liquid stored in the reservoir 110.

The spirit of the present disclosure should not be determined by being limited to the above-described embodiments, and not only the claims to be described later, but also any range equivalent to or equivalently changed from the claims falls within the scope of the spirit of the present disclosure.

1. A reservoir assembly comprising:
a reservoir comprising a storage space for medical liquid;
a plunger inserted into the reservoir and moving along the reservoir;
a connector connected to the plunger and comprising at least a portion extending to an opposite side of the storage space;
a guide member into which the connector is inserted and which guides movement of the connector; and
a sensor unit installed on the guide member and sensing movement of the connector.

2. The reservoir assembly of claim 1, wherein the connector is flexible.

3. The reservoir assembly of claim 1, wherein the guide member comprises:
a first end arranged on the reservoir to face the plunger;
a second end arranged on a side surface of the reservoir; and
a connection portion connecting the first end to the second end and comprising a curved section.

4. The reservoir assembly of claim 3, wherein the sensor unit comprises a plurality of contact terminals which are arranged separated from each other on the second end.

5. The reservoir assembly of claim 1, wherein the sensor unit comprises a plurality of contact terminals and generates a signal when the connector contacts the plurality of contact terminals.

6. The reservoir assembly of claim 5, wherein at least one of the plurality of contact terminals is arranged to rotate along linear movement of the connector.

7. The reservoir assembly of claim 1, wherein the connector comprises:
a concave first surface; and
a convex second surface arranged on an opposite side to the concave first surface.

8. The reservoir assembly of claim 7, wherein the concave first surface is arranged inside a curved section of the guide member, and a second surface is arranged outside the curved section of the guide member.

9. A medical liquid injection device comprising:
a reservoir assembly in which medical liquid is stored;
a needle assembly ejecting the medical liquid; and
a driving unit connected to the reservoir assembly and moving the medical liquid to the needle assembly from the reservoir assembly in driving,
wherein the reservoir assembly comprises:
a reservoir comprising a storage space for the medical liquid;
a plunger inserted into the reservoir and moving along the reservoir;
a connector connected to the plunger and comprising at least a portion extending to an opposite side of the storage space;
a guide member into which the connector is inserted and which guides movement of the connector; and
a sensor unit installed on the guide member and contacting the connector along movement of the connector.
